(19) United States
(12) Patent Application Publication
(10) Pub. No.: US 2022/0235411 A1
(43) Pub. Date: Jul. 28, 2022

(54) METHOD OF ANALYZING NUCLEIC ACID TEMPLATES USING RAPID LAMP

(71) Applicants: Samuel WILLIAMS, New York, NY (US); Kristina BRAZHNIK, Bronx, NY (US)

(72) Inventors: Samuel WILLIAMS, New York, NY (US); Kristina BRAZHNIK, Bronx, NY (US)

(21) Appl. No.: 17/300,304

(22) Filed: May 28, 2021

Related U.S. Application Data
(63) Continuation of application No. 15/788,791, filed on Oct. 19, 2017, now abandoned.
(60) Provisional application No. 62/410,362, filed on Oct. 19, 2016.

(51) Int. Cl. C12Q 1/6851; C12Q 1/6888; C12Q 1/6879

(52) U.S. Cl. CPC C12Q 1/6851; C12Q 2600/16; C12Q 1/6879; C12Q 1/6888

(57) ABSTRACT

Disclosed herein are methods to determine the abundance of nucleotide sequences relative to other nucleotide sequences in a complex genome.

### CROSS REFERENCE TO RELATED APPLICATIONS

This application is a continuation of U.S. application Ser. No. 15/788,791, filed Oct. 19, 2017, which claims the benefit of U.S. Provisional Application No. 62/410,362, filed Oct. 19, 2016, the disclosures of which are incorporated herein in their entireties.

### INCORPORATION OF SEQUENCE LISTING

The sequence listing that is contained in the file named “LAV0002201US_ST25,” which is 8.89 kilobytes as measured in Microsoft Windows operating system and was created on Jan. 18, 2018, is filed electronically herewith and incorporated herein by reference.

### FIELD OF THE DISCLOSURE

Disclosed herein are methods to determine the abundance of nucleotide sequences relative to other nucleotide sequences in a complex genome.

### BACKGROUND OF THE DISCLOSURE

Chromosomal integrity is the overriding determinant of embryonic, fetal, and neonatal morbidity and mortality. Consequently, testing for aneuploidy, copy number variations (CNVs), and genetic sex is critical for pre-implantation genetic screening (PGS) of embryos produced by in vitro fertilization (IVF), prenatal testing, testing in neonates with cardiac or morphologic anomalies and ambiguous genitalia, and evaluation of products-of-conception following a pregnancy loss.

Existing methods to test for aneuploidy, CNVs, and genetic sex, include G-band karyotyping, multiplex ligation-dependent probe amplification (MLPA), microarray analysis, fluorescence in situ hybridization (FISH), and next generation sequencing (NGS). Unfortunately, these methods require a centralized high-complexity laboratory, are expensive, and cannot be performed in resource-poor settings. Additionally, they require over 24 hours to perform, thereby increasing anxiety and potentially delaying—or even preventing—diagnosis and treatment.

Loop-mediated isothermal amplification (LAMP) is a relatively new technique that rapidly amplifies specific DNA targets under isothermal conditions in a one-step process using specific primers and a DNA polymerase with high strand displacement and replication activity. By coupling DNA amplification to a reporter system, the presence of a target sequence can be detected. The reaction occurs under a constant temperature (typically 60-65° C.), obviating the need for costly thermocyclers. Due to the nature of the amplification reaction and without the need to cycle between reaction temperatures, amplification occurs quickly, in as little as 10-15 minutes. Since primers are used to recognize six different DNA target sequences, the reaction is exquisitely specific.

Because the system is robust, rapid and low-cost, multiple LAMP-based assays have been developed for point-of-care detection of microorganisms including viruses, bacteria and fungi, food quality control, tumor detection and sexing of bovine embryos and forensic samples. The present disclosure describes a quantitative LAMP-based assay (qLAMP) and demonstrates its application for sexing of human embryos and determination of aneuploidy in human clinical samples.

### SUMMARY OF THE DISCLOSURE

In one embodiment, the disclosure describes a method of analyzing one or more nucleic acid templates, comprising: a) providing a multiplex reaction mixture comprising: (i) at least one nucleic acid template to be amplified, (ii) at least one nucleic acid target, (iii) primers for amplifying the nucleic acid template, and (iv) primers for amplifying the nucleic acid target; b) co-amplifying the complex template nucleic acid and the nucleic acid target with a polymerase, wherein the co-amplification takes place isothermally and comprises a heat pulse step of at least 94° C., wherein nucleic acid template and nucleic acid target are amplified; c) normalizing the nucleic acid template, wherein the normalization is carried out after completion of the co-amplification of step b).

In another embodiment, the quantity of the total amplified control nucleic acid and the quantity of total nucleic acid template are correlated with a completed amplification of the nucleic acid template, wherein the co-amplifying step is done isothermally.

In yet another embodiment, the isothermal co-amplifying step includes, but is not limited to, nucleic acid sequence-based amplification (NASBA), loop-mediated amplification (LAMP), helicase-dependent amplification (HDA), rolling circle amplification (RCA), recombinase polymerase amplification (RPA), and multiple displacement amplification (MDA).

In another embodiment, the methods disclosed herein, can be used for testing for aneuploidy, copy number variations (CNVs), and genetic sex.

In another embodiment, the methods disclosed herein, can be used for pathogen detection.

In another embodiment, the one or more nucleic acid template and/or the nucleic acid target is a human chromosome.

In yet another embodiment the human chromosome is Chromosome 1 or Chromosome 21.

In another embodiment, the polymerase concentration is from about 0.28 to about 0.56 units/μL. In another embodiment, the Tm is from about 60° C. to about 65° C. for qLAMP, or from about 57° C. to about 72° C. for nested PCR (NEST). In another embodiment, the primer concentration is from about 25 nM to about 1600 nM. In another embodiment, the magnesium concentration is from about 5 mM to about 8 mM. In another embodiment, the template DNA concentration is less than 0.1 ng. In another embodiment, a 1.5-fold difference in target DNA concentration is detected. In another embodiment, the method further comprises a plurality of primers for amplifying a plurality of nucleic acid targets and a plurality of nucleic acid templates. In some embodiments, the concentrations of the F3/B3 primer pair are from about 25 nM to about 200 nM. In another embodiment, the concentrations of the LF/LB primer pair are from about 50 nM to about 400 nM. In another embodiment, the concentration of the BIP primer pair is from about 200 nM to about 1600 nM. In another embodiment, the concentration of the FIP primer pair is from about 200 nM to about 1600 nM. In another embodiment, the FIP primer pair concentration is from about 200 nM to about 1600 nM in SyBr Green based reactions. In another embodiment, the concentration of the FIP primer pair is from about 100 nM to about 800 nM. In another embodiment, the FIP primer pair concentration is from about 100 nM to about 800 nM in probe based reactions. In another embodiment, the probe based reaction comprises the presence of from about 100 nM to about 800 nM FIP FQ-Fd duplex.

### BRIEF DESCRIPTION OF THE SEQUENCES

SEQ ID NO:1—Sequence of FIP primer for human unique Y-chromosome target (SRY gene [ID 6736, NCBI]).

SEQ ID NO:2—Sequence of BIP primer for human unique Y-chromosome target (SRY gene [ID 6736, NCBI])

SEQ ID NO:3—Sequence of F3 primer for human unique Y-chromosome target (SRY gene [ID 6736, NCBI])

SEQ ID NO:4—Sequence of B3 primer for human unique Y-chromosome target (SRY gene [ID 6736, NCBI])

SEQ ID NO:5—Sequence of LF primer for human unique Y-chromosome target (SRY gene [ID 6736, NCBI])

SEQ ID NO:6—Sequence of LB primer for human unique Y-chromosome target (SRY gene [ID 6736, NCBI])

SEQ ID NO:7—Sequence of FIP primer for human unique X chromosome target (RPA4 gene [ID29935, NCBI])

SEQ ID NO:8—Sequence of BIP primer for human unique X chromosome target (RPA4 gene [ID29935, NCBI])

SEQ ID NO:9—Sequence of F3 primer for human unique X chromosome target (RPA4 gene [ID29935, NCBI])

SEQ ID NO:10—Sequence of B3 primer for human unique X chromosome target (RPA4 gene [ID29935, NCBI])

SEQ ID NO:11—Sequence of LF primer for human unique X chromosome target (RPA4 gene [ID29935, NCBI])

SEQ ID NO:12—Sequence of LB primer for human unique X chromosome target (RPA4 gene [ID29935, NCBI])

SEQ ID NO:13—Sequence of FIP primer for human unique Reference target (CFTR gene [ID 1080, NCBI])

SEQ ID NO:14—Sequence of BIP primer for human unique Reference target (CFTR gene [ID 1080, NCBI])

SEQ ID NO:15—Sequence of F3 primer for human unique Reference target (CFTR gene [ID 1080, NCBI])

SEQ ID NO:16—Sequence of B3 primer for human unique Reference target (CFTR gene [ID 1080, NCBI])

SEQ ID NO:17—Sequence of LF primer for human unique Reference target (CFTR gene [ID 1080, NCBI])

SEQ ID NO:18—Sequence of LB primer for human unique Reference target (CFTR gene [ID 1080, NCBI])

SEQ ID NO:19—Sequence of hSRY-5IABkFQ-FIP oligonucleotide probe specific for human unique Y-chromosome target (SRY gene).

SEQ ID NO:20—Sequence of hSRY-36-FAM-Fd oligonucleotide probe specific for human unique Y-chromosome target (SRY gene).

SEQ ID NO:21—Sequence of hRPA4-5IABkFQ-FIP oligonucleotide probe specific for human unique X-chromosome target (RPA4 gene).

SEQ ID NO:22—Sequence of hRPA4-3JOE_N-Fd oligonucleotide probe specific for human unique X-chromosome target (RPA4 gene).

SEQ ID NO:23—Sequence of hRPA4-36-FAM-Fd oligonucleotide probe specific for human unique X-chromosome target (RPA4 gene).

SEQ ID NO:24—Sequence of hCFTR-5IABkFQ-FIP oligonucleotide probe specific for human reference target (CFTR gene).

SEQ ID NO:25—Sequence of hCFTR-36-TAMSp-Fd oligonucleotide probe specific for human reference target (CFTR gene).

SEQ ID NO:26-60—Primers used for multiplex qLAMP in addition to RPA4 and SRY primers.

### BRIEF DESCRIPTION OF THE DRAWINGS

FIG. 1A: real-time amplification plots for LAMP-mediated generation of SRY target product (Y chromosome) in model DNA sample from 46 XY blood and absence of the amplification signal in DNA sample from 46 XX blood. FIG. 1B: Real-time amplification plots for LAMP-mediated generation of RPA4 target product (X chromosome) in model DNA samples from 46 XY and 46 XX blood. FIG. 1C: Real-time amplification plots for LAMP-mediated generation of SRY target product (Y chromosome) in AF samples corresponding to embryo of different sex karyotype and processed with alternative methods. AF 1 (46 XX): AF 1.1—frozen/thawed sample; AF 1.2—sonicated sample. AF 2 (46 XY): AF 2.1—frozen/thawed sample; AF 2.2—sonicated sample; AF 2.3—lysed/neutralized sample. FIG. 1D: Comparison of model 46 XY vs. 46 XX for SRY (Y chromosome) and RPA4 (X chromosome) targets amplification.

FIG. 2A: Real-time amplification plots for LAMP-mediated generation of SRY target product (Y chromosome) in 2-fold serial dilutions of isolated SRY target. FIG. 2B: Dilution curve plotted based on Ct values, e.g., time to detect respective positive amplification signal (threshold ΔRn) in 2-fold serial dilutions of isolated SRY target. Each point dilution was analyzed in triplicates.

FIG. 3A-FIG. 3D. Quantitative LAMP-based detection of intact SRY target (Y chromosome) within whole genome content in 1.5-fold serial dilutions of human genomic DNA sample (46 XY) isolated from blood cells. Dilution curves plotted based on Ct values, e.g., time to detect respective positive amplification signal (threshold ΔRn) in 1.5-fold serial dilutions of either original or pre-heated human genomic DNA (46 XY). FIG. 3A: Original (untreated) DNA. FIG. 3B: Genomic DNA pre-heated for 60 sec at 100° C. FIG. 3C: Genomic DNA pre-heated for 90 sec at 100° C. FIG. 3D: Comparison of dilution curves plotted for 1.5-fold serial dilutions of original and pre-heated genomic DNA. Each point dilution was analyzed in triplicates.

FIG. 4A-FIG. 4B. LAMP-based quantification of intact RPA4 target (X chromosome) copy number ratio in 2-fold serial dilutions of human genomic DNA samples of different sex karyotypes (46 XY vs. 46 XX) isolated from primary blood cells. FIG. 4A: Dilution curves plotted based on Ct values, e.g., time to detect respective positive signal of target amplification (threshold ΔRn) in 2-fold serial dilutions of human genomic DNA (46 XY vs. 46 XX). FIG. 4B: Comparison of calculated and normalized copy number ratio of RPA4 target, e.g., X chromosome, in 2-fold serial dilutions of DNA samples (46 XY (reference sample to normalize to) vs. 46 XX) isolated from primary blood cells. Each point dilution was analyzed in triplicates and presented data is the average result of two independent experiments.

FIG. 5A and FIG. 5B show quantitative LAMP-based detection of pre-amplified SRY target (Y chromosome) isolated out of genome content in 10-fold serial sample dilutions. FIG. 5A: shows real-time amplification plots for LAMP-mediated generation of SRY target product (Y chromosome) in 10-fold serial dilutions of isolated SRY target. FIG. 5B: shows dilution curve plotted based on Ct values, e.g., time to detect respective positive amplification signal (threshold ΔRn) in 10-fold serial dilutions of isolated SRY target. Each point dilution was analyzed in triplicate.

FIG. 6. Quantitative LAMP-based detection of pre-amplified SRY target (Y chromosome) isolated out of genome content in 2-fold serial sample dilutions in the absence (square) and presence (circle) of SRY target-free 46 XX genomic DNA. Dilution curve plotted based on Ct values in 2-fold serial dilutions of isolated SRY target. Each point dilution was analyzed in triplicates.

FIG. 7. Quantitative LAMP-based detection of pre-amplified SRY target (Y chromosome) isolated out of genome content in 1.5-fold serial sample dilutions. Dilution curve plotted based on Ct values in 1.5-fold serial dilutions of isolated SRY target. Each point dilution was analyzed in triplicates.

FIG. 8A through FIG. 8C show real-time amplification plots for LAMP-mediated generation of SRY target product (Y chromosome) in 2-fold serial dilutions of original non-treated and enzymatically digested genomic 46 XY DNA. FIG. 8A: Original non-treated DNA; FIG. 8B: Genomic DNA digested for 10 min; FIG. 8C: Genomic DNA digested for 20 min. Each point dilution was analyzed in triplicate.

FIG. 9A through FIG. 9D show LAMP of SRY target (Y chromosome) in 2-fold serial dilutions of original non-treated and mechanically digested (needle-shearing) genomic 46 XY DNA results in poor-quality quantification. FIG. 9A: Real-time amplification plots for 2-fold dilutions of original non-treated DNA. FIG. 9B: Dilution curve plotted based on Ct values for 2-fold dilutions of original non-treated DNA. FIG. 9C: Real-time amplification plots for 2-fold dilutions of mechanically digested DNA (shearing by passing through insulin needle 20 times up and down). FIG. 9D: Dilution curve plotted based on Ct values for 2-fold dilutions of mechanically digested DNA (shearing by passing through insulin needle 20 times up and down). Each point dilution was analyzed in triplicates.

FIG. 10. LAMP-based quantification of intact RPA4 target (X chromosome) copy number ratio in 2-fold serial dilutions of pre-heated human genomic DNA samples of different sex karyotypes (46 XY vs. 46 XX) isolated from primary blood and cultured cells. Comparison of dilution curves plotted based on Ct values, e.g., time to detect respective positive signal of target amplification (threshold ΔRn) in 2-fold serial dilution points for DNA of different origin (blood or cultured cells). Each point dilution was analyzed in triplicates.

### DETAILED DESCRIPTION OF THE DISCLOSURE

The present disclosure provides a method of analyzing one or more nucleic acid templates, comprising: a) providing a multiplex reaction mixture comprising: (i) at least one nucleic acid template to be amplified, (ii) at least one nucleic acid target, (iii) primers for amplifying the nucleic acid template, and (iv) primers for amplifying the nucleic acid target; b) co-amplifying the complex template nucleic acid and the nucleic acid target with a polymerase, wherein the co-amplification takes place isothermally and comprises a heat pulse step of at least 94° C., wherein nucleic acid template and nucleic acid target are amplified; c) normalizing the nucleic acid template, wherein the normalization is carried out after completion of the co-amplification of step b).

Rapid, affordable, point-of-care testing for genetic abnormalities has been a longstanding goal for molecular diagnostics in pre-implantation, prenatal and postnatal diagnostics. Existing methods for chromosomal assessment are time-consuming, costly, and require a centralized high-complexity laboratory. Consequently, diagnosis and treatment is delayed and testing is often not an option in resource-poor settings.

The inventors aimed to develop a rapid, inexpensive test that can be performed at point-of-care for the detection of chromosomal aneuploidy. Here, Quantitative Loop-mediated isothermal amplification (qLAMP), previously used for qualitative analysis of target DNA sequences, was adapted for quantitative detection of chromosomal aneuploidy. A reporter assay was optimized and target-specific fluorescent probes were used that enable real-time monitoring of fluorescence increase during product amplification. Peripheral blood, cell culture, and amniotic fluid samples were then tested using six primer sets for two unique single-copy target genes: SRY and RPA4 genes on chromosomes Y and X, respectively.

Under these conditions, it was possible to readily distinguish between male and female samples and to identify 46 XX, 46 XY, 47 XYY and 45 XO karyotypes within 20 minutes, using as little as 1 ng of genomic DNA. Thus, qLAMP can provide assessment of relative copy numbers of genomic DNA targets in under 20 minutes without the need for complex laboratory equipment or training. When optimized and adapted, this assay may enable accurate screening for CNVs and aneuploidy for all chromosomes.

In accordance with the disclosure, a nucleic acid template may be DNA or RNA. For example, in some embodiments, a nucleic acid template may be a nucleic acid selected from the group consisting of cDNA (complementary DNA), LNA (locked nucleic acid), mRNA (messenger RNA), mtRNA (mitochondrial RNA), rRNA (ribosomal RNA), tRNA (transfer RNA), nRNA (nuclear RNA), dsRNA (double-stranded RNA), ribozyme, riboswitch, viral RNA, dsDNA (double-stranded DNA), ssDNA (single-stranded DNA), plasmid DNA, cosmid DNA, chromosomal DNA, viral DNA, mtDNA (mitochondrial DNA), nDNA (nuclear DNA). In some embodiments, a nucleic acid template may be more than one of such nucleic acid types. Furthermore, the nucleic acid template can also be the entirety of a group of nucleic acids, preferably the entirety of mRNA or cDNA (transcriptome), respectively, and/or the entirety of DNA (genome) of one or more organisms. A person skilled in the art knows that the entirety of RNA can represent the genome of an organism, which, in particular, may apply to RNA viruses. A nucleic acid template as described herein may also be one or more chromosomes.

A nucleic acid template to be analyzed may have different origins. For example, a nucleic acid template may be isolated from one or more organisms selected from the group consisting of viruses, phages, bacteria, eukaryotes, plants, fungi, and animals. In some embodiments, a nucleic acid template may be from a particular cell type, such as including, but not limited to, blood cells, or immune cells, such as B or T lymphocytes. Other embodiments provide samples isolated or originating from any cell line, such as a fibroblast cell line. Any particular cell type or line may be used. Furthermore, the nucleic acid template to be analyzed can be a part or a portion of a particular sample. Such samples may also be of various origins. Thus, the method of the disclosure may also relate to the analysis of nucleic acids contained in, for example, an environmental sample, such as a water sample. In some embodiments, it is envisioned that a sample may be obtained from any source from which a nucleic acid template may be obtained, including, but not limited to, a biological sample, such as a blood sample, a serum sample, an amniotic fluid sample, cerebrospinal fluid sample, or the like.

As used herein, an isothermal reaction is intended to refer to a reaction that is carried out only at one temperature. If the temperature of the reaction is changed prior to the beginning of the reaction (e.g., incubation, storage, or transport on ice or in refrigerated conditions) or after completion of the reaction (e.g., in order to inactivate reaction components or enzymes), the reaction is still referred to as isothermal, as long as the reaction per se is carried out at a constant temperature. The temperature is considered to be constant if the temperature variations do not exceed +/−10° C.

### I. Chromosome-Specific Targets and LAMP Primers Design

Two unique single-copy target genes with invariant loci were identified and selected for each of the human sex chromosomes through mining both literature and databases (International HapMap Project, Entrez SNP, DECIPHER (Database of Chromosomal Imbalance and Phenotype in Humans using Ensembl Resources, Decipher Consortium)): SRY (sex determining region Y [ID 6736, NCBI]), specific for the Y chromosome and RPA4 (replication protein A4 [ID29935, NCBI]), specific for the X chromosome. LAMP primer sets of combinations of six primers (FIP, BIP, F3, B3, LF, LB) for each of the target genes were designed with the aid of LAMP primer designing software PrimerExplorer V4. Primer design and optimization is well known in the art. While the primers as described herein were used to demonstrate the methods of the present disclosure, the methods are not intended to be limited to a particular primer sequence. One of skill in the art would recognize that other software programs and/or primer sequences may be used with similar successful results without deviating from the scope of the disclosure.

TABLE 1
LAMP primers used for human unique Y-
and X-chromosome target genes, and
reference target gene (CFTR).

| | |
|---|---|
| Probe | Sequence (5′-3′) |

Y chromosome target (SRY gene [ID 6736, NCBI])

| | |
|---|---|
| FIP | TCTGCGGGAAGCAAACTGCAATA |
| | AGTATCGACCTCGTCGGAA |
| | (SEQ ID NO: 1) |
| BIP | CCGCTTCGGTACTCTGCAGCTTG |
| | AGTGTGTGGCTTTCGTA |
| | (SEQ ID NO: 2) |
| F3 | AGGCCATGCACAGAGAGAA |
| | (SEQ ID NO:3) |
| B3 | CCTAGCTGGTGCTCCATTC |
| | (SEQ ID NO:4) |
| LF | TCGGCAGCATCTTCGCC |
| | (SEQ ID NO:5) |
| LB | GAAGTGCAACTGGACAACAGG |
| | (SEQ ID N0:6) |

X chromosome target (RPA4 gene [ID29935, NCBI])

| | |
|---|---|
| FIP | CGGAGCTCATGGATGCTCTTC |
| | CCGCAATTTCATCCAGGACGA |
| | (SEQ ID NO: 7) |
| BIP | GGCTCAGCTCTGCGACCTTAG |
| | TAGATGTGGCCCTCAACGG |
| | (SEQ ID NO: 8) |
| F3 | GCTGGGGATAACGATGAGAG |
| | (SEQ ID N0:9) |
| B3 | TGCTCCCGATCCACAGTG |
| | (SEQ ID NO: 10) |
| LF | CTCATGAATCAAACGCAGC |
| | ACT (SEQ ID NO: 11) |
| LB | CGTCAAGGCCATCAAGGAAG |
| | (SEQ ID NO: 12) |

Reference target (CFTR gene [ID 1080, NCBI])

| | |
|---|---|
| FIP | CCAAAGAGTAAAGTCCTTC |
| | TCTCTCGAGAGACTGTTGG |
| | CCCTTGAAGG |
| | (SEQ ID NO. 13) |
| BIP | GTGTTGATGTTATCCACC |
| | TTTTGTGGACTAGGAAAA |
| | CAGATCAATAG |
| | (SEQ ID NO: 14) |
| F3 | TAATCCTGGAACTCCGGT |
| | GC (SEQ ID NO: 15) |
| B3 | TTTATGCCAATTAACATT |
| | TTGAC |
| | (SEQ ID NO: 16) |
| LF | ATCCACAGGGAGGAGCTCT |
| | (SEQ ID NO: 17) |
| LB | CTCCACCTATAAAATCGGC |
| | (SEQ ID NO: 18) |

LAMP primers for CFTR (cystic fibrosis transmembrane conductance regulator [ID 1080, NCBI]) gene target used as internal reference control for signal normalization were published elsewhere.

Additional probes represented by a fluorescently labeled FIP primer aligned together with a complementary Fd oligo linked to a quencher were designed and synthesized to ensure target-specific, fluorescence-based read-out to detect and discriminate each unique target in singleplex/multiplex assays. Primer sequences are provided in Table 2.

TABLE 2
Oligonucleotide-probe pairs labeled with
fluorescent dye or quencher specific for
human unique Y- and X-chroinosome target
genes and reference target gene (CFTR).

| | Sequence | Posi- | Modifi- |
|---|---|---|---|
| Probe | (5′-3′) | tion | cation |

Y chromosome target (SRY gene [ID 6736, NCBI])

| | | | |
|---|---|---|---|
| hSRY-5IABkFQ- | TCTGCGGGAAGCA | 5′ | Iowa |
| FIP | AACTGCAATAAGT | | Black® |
| | ATCGACCTCGTCG | | FQ |
| | GAA | | |
| | (SEQ ID NO: 19) | | |
| hSRY-36-FAM- | ATTGCAGTTTGCT | 3′ | 6-FAM |
| Fd | TCCCGCAGA | | |
| | (SEQ ID NO: 20) | | |

X chromosome target (RPA4 gene [ID29935, NCBI])

| | | | |
|---|---|---|---|
| hRPA4-5IABkFQ- | CGGAGCTCATGGA | 5′ | Iowa |
| FIP | TGCTCTTCCCGCA | | Black® |
| | ATTTCATCCAGGA | | FQ |
| | CGA | | |
| | (SEQ ID NO: 21) | | |
| hRPA4-3JOE_N- | GGAAGAGCATCCA | 3′ | JOE (NHS |
| Fd | TGAGCTCC | | ester) |
| | (SEQ ID NO: 22) | | |
| hRPA4-36-FAM-Fd | GGAAGAGCATCCA | 3′ | 6-FAM |
| | TGAGCTCC | | |
| | (SEQ ID NO: 23) | | |

Reference target (CFTR gene [ID 1080, NCBI])

| | | | |
|---|---|---|---|
| hCFTR-5IABkFQ- | CCAAAGAGTAAAG | 5′ | Iowa |
| FIP | TCCTTCTCTCTCG | | Black® |
| | AGAGACTGTTGGC | | FQ |
| | CCTTGAAGG | | |
| | (SEQ ID NO: 24) | | |
| hCFTR-36-TAMSp- | AGAGAGAAGGAC | 3′ | TAMRA™ |
| Fd | TTTACTCTTT | | |
| | (SEQ ID NO: 25) | | |

### II. Target DNA Samples

For initial development of qLAMP, a 200-bp PCR product from the human SRY gene (sex determining region Y [ID 6736, NCBI]) and RPA4 gene (replication protein A4 [ID29935, NCBI]) was purified using QIAquick PCR purification kit (QIAGEN) followed by confirmation by Sanger was used as a target sequence. Primer sequences used are provided in Table 2. Any unique target gene may be used in accordance with the present disclosure, as long as the selected target gene provides specific identification for the particular application. Single-copy-number genes may provide beneficial results, or sufficiently low-copy-number genes may also be used, depending on the particular use. Any concentration of the target gene may be used, as described herein. In some embodiments, low concentrations of target gene nucleic acid and/or the amplification products thereof may be beneficial in cases where there is minimal nucleic sample for analysis. Serial dilutions may be made as necessary to provide a desired concentration of the target nucleic acid sequence. For example, for the presently described SRY and RPA4 analyses, serial dilutions of the purified PCR fragments were initially tested to optimize the LAMP parameters for correct quantification and to characterize test limitations, as described herein.

Subsequent testing on human genomic samples utilized DNA samples isolated from primary blood cells of adults and well-characterized lymphoblastoid cell lines from B-lymphocytes with normal male (46 XY) [GM12877, Coriell] and female (46 XX) [GM12878 Coriell] karyotypes. Samples with abnormal ratios of the sex chromosomes (47 XYY, 45 XO) originated from respective cell line of Coriell Cell Repository (e.g., fibroblast cell line, XYY syndrome [GM11337]) and from original human tissue [specify what type of samples] samples. Intact whole genome DNA was isolated from tissues and blood or cultured cells by means of appropriate column-based manufacture protocols (QIAamp® DNA Mini kit, QIAamp® DNA Blood Mini/Midi kits, QIAGEN). Amniotic fluid (AF) samples were collected from pregnant patients and genomic DNA was isolated using QIAamp® DNA Mini kit (QIAGEN). Genomic DNA was isolated based on different protocols including freezing (−80° C.) and thawing (37° C.) cycles, adding equal volumes of lysis buffer, and sonication (3-10 sec at stage 1-2 intensity level (Microson™ XL2000, Qsonica, LLC)).

As would be understood by one of skill in the art, amplification of nucleic acid targets or templates may require preparation of the sample, for example, to inactivate particular enzymes or proteins, to degrade a particular nucleic acid, or to release secondary structure from genomic DNA. Any particular method for such sample preparation may be used with the present methods, such as incubation of samples at a particular temperature, or the addition of certain detergents or reagents. For the present case, samples were incubated at 100° C. for various times ranging from 30-120 sec. Both untreated intact genomic DNA (control) and pre-heated DNA were used to prepare 10-, 2- and 1.5-fold serial dilutions and testing was performed on these using LAMP for specific target quantification, as described herein.

### III. LAMP Reaction

In some embodiments, LAMP analysis as described herein may be performed using target-specific fluorescent probes (e.g., 5′-Quencher-FIP:Fd-Fluorophore-3′ (Q-FIP:Fd-Fluo) duplex), to enable specific and sensitive detection of the target gene. Such a target-specific probe may be prepared, for example, immediately prior to use by mixing equal volumes of 200 μM 5′-Quencher-FIP (5′-modification) and 200 μM Fd-Fluorophore (3′-modification) and double-volume of nuclease-free water to reach a desired concentration. Any particular concentration of a target-specific probe and/or an oligonucleotide as described herein may be useful in accordance with the present disclosure, such as including, but not limited to, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 150 μM, 200 μM, 250 μM, 300 μM, or the like. In some embodiments, a 50 μM concentration of each oligonucleotide may provide beneficial results. One of skill in the art will recognize that the concentration of a target-specific probe and/or oligonucleotide may be optimized as necessary to obtain the desired results. In the present case, the mixture was heated for 3 min at 98° C. to align complementary sequences, followed by slow gradual cooling to room temperature.

A complete LAMP reaction or reaction mix with one or more target-specific fluorescent probes may contain any desired concentration of each component and/or reagent to achieve the desired results. Such individual components may be individually or separately optimized for this purpose. For example, as described in detail herein and in the Examples, a LAMP reaction of the present disclosure may contain, for example, 0.8 μM FIP and 0.4 μM Q-FIP:Fd-Fluo duplex, 1.6 μM BIP, 0.2 μM each of F3 and B3, 0.4 μM each of LF and LB; 2.8 U Bst 2.0 WarmStart DNA polymerase (New England Biolabs (NEB)); 1 mM each of dNTPs (NEB); DNA template (tested dilution); 1× Isothermal Amplification Buffer additionally supplemented with 6 mM MgSO₄ (NEB), 1×ROX reference dye (0.5 μM). For multiplex reactions, total primer concentrations may also be optimized as necessary for the individual assay. For multiplex assays or reactions, concentrations of reagents may be kept as for single assays, or may be altered to suit the particular application. The present disclosure is intended to encompass any particular concentrations that may provide the desired results. In some embodiments, concentrations of reagents may be used as described herein for a standard LAMP reaction, with each set of primers and/or probes representing 1/n of the total, where n is the number of targets and respective primer sets being evaluated in a particular analysis.

In some embodiments, concentrations of reagents for a LAMP assay as described herein may be any concentration appropriate for the particular use. For example, non-limiting examples of concentrations that may be used in a LAMP assay of the present disclosure may include, but are not limited to, a polymerase concentration from about 0.28 to about 0.56 units/μL. The polymerase may be any polymerase capable of amplification required to achieve the desired results. In another embodiment, the Tm is from about 60° C. to about 65° C. In another embodiment, the primer concentration is from about 25 nM to about 1600 nM. In another embodiment, the magnesium concentration is from about 5 mM to about 8 mM. In another embodiment, the template DNA concentration is less than 0.1 ng. In another embodiment, a 1.5-fold difference in target DNA concentration is detected. In another embodiment, the method further comprises a plurality of primers for amplifying a plurality of nucleic acid targets and a plurality of nucleic acid templates. In some embodiments, the concentrations of the F3/B3 primer pair are from about 25 nM to about 200 nM. In another embodiment, the concentrations of the LF/LB primer pair are from about 50 nM to about 400 nM. In another embodiment, the concentration of the BIP primer pair is from about 200 nM to about 1600 nM. In another embodiment, the concentration of the FIP primer pair is from about 200 nM to about 1600 nM. In another embodiment, the FIP primer pair concentration is from about 200 nM to about 1600 nM in SyBr Green-based reactions. In another embodiment, the concentration of the FIP primer pair is from about 100 nM to about 800 nM. In another embodiment, the FIP primer pair concentration is from about 100 nM to about 800 nM in probe based reactions. In another embodiment, the probe based reaction comprises the presence of from about 100 nM to about 800 nM FIP FQ-Fd duplex.

LAMP reactions may be performed in any reaction volume, for example including, but not limited to, 5 μl, 10 μl, 15 μl, 20 μl, 25 μl, 30 μl, 35 μl, 40 μl, 45 μl, 50 μl, 60 μl, 70 μl, 80 μl, 90 μl, 100 μl, 125 μl, 150 μl, 175 μl, 200 μl, 250 μl, 300 μl, 350 μl, 400 μl, 450 μl, 500 μl, 1 mL, or the like. In some particular applications, it may be beneficial to perform reactions in duplicate or triplicate within an experiment (intra-assay) and in independent experiments (inter-assay). For the analyses, the reaction plate may be incubated at any desired temperature, or at any temperature range appropriate for the particular application. In the present case, the reaction plate was incubated at 62° C. for 60 min and monitored in real-time mode using qPCR real-time cycler (StepOnePlus™ Real-Time PCR Systems, Applied Biosystems) with 30-sec cycling steps.

### IV. Data Analysis, Statistics

As described herein, analysis of the data may be performed using any applicable statistical methods, such as Ct value, in order to reflect the time taken to reach a positive signal threshold. These values may be used to plot calibration curves as a function of target copy number input load for each separate target in the reference sample (46 XY karyotype). Respective amplification Ct values for each dilution of test samples may be used to calculate input target copy number in those samples. The obtained target copy number in each dilution of test sample may be normalized to a respective value for a reference sample dilution in order to calculate target copy number ratio.

Means and variances of the rates of concurrence may be evaluated for significance with the Student's t-test with determination of effect size along with p-values and standard deviations within each experiment for duplicates and triplicates (intra-assay) and between independent experiments (inter-assay).

### V. Modification of Nucleic Acids

In some embodiments, it may be useful to modify or synthesize a nucleic acid target or template sequence or molecule. Any number of methods well known to those skilled in the art can be used to isolate and manipulate a DNA molecule. For example, PCR technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules are capable of forming an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Stringency conditions are known in the art and would be understood by one of skill reading the present disclosure. One of skill in the art will also understand that stringency may be altered as appropriate to ensure optimum results. Complementarity as described herein also refers to the binding of a DNA editing enzyme to its target in vivo or in vitro. One of skill in the art would recognize that variations in complementarity will depend on the particular nucleic acid sequence and will be able to modify conditions as appropriate to account for this.

Polynucleotides of the present disclosure can be composed of either RNA or DNA. Preferably, the polynucleotides are composed of DNA. The subject disclosure also encompasses those polynucleotides that are complementary in sequence to the polynucleotides disclosed herein. Polynucleotides and polypeptides of the disclosure can be provided in purified or isolated form.

Because of the degeneracy of the genetic code, a variety of different polynucleotide sequences can encode polypeptides of the present disclosure. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides of the subject disclosure. These variant or alternative polynucleotide sequences are within the scope of the subject disclosure. As used herein, references to “essentially the same” sequence refers to sequences which encode amino acid substitutions, deletions, additions, or insertions which do not materially alter the functional activity of the polypeptide encoded by the polynucleotides of the present disclosure.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotides at every position are the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

Nucleic acid target molecules and nucleic acid template molecules as described herein may refer to polynucleotides that can be defined in terms of identity and/or similarity ranges with those sequences of the disclosure specifically exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

As used herein, the terms “nucleic acid” and “polynucleotide” refer to a deoxyribonucleotide, ribonucleotide, or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include the DNA strand sequence that is transcribed into RNA and the strand sequence that is complementary to the DNA strand that is transcribed. The polynucleotide sequences also include both full-length sequences as well as shorter sequences derived from the full-length sequences. The polynucleotide sequence includes both the sense and antisense strands either as individual strands or in the duplex.

### VI. Kits

The disclosure further provides a kit comprising a single-use container comprising one or more components for performing LAMP analyses as described herein. A kit may further comprise reagents for nucleic acid isolation, PCR, or other methods that may be useful in accordance with the present disclosure.

Components provided in a kit of the disclosure may include, for example, any starting materials useful for performing a method as described herein. Such a kit may comprise one or more such reagents or components for use in a variety of assays, including for example, nucleic acid assays, e.g., PCR or RT-PCR assays, genetic complementation assays, or any assay useful in accordance with the disclosure. Components may be provided in lyophilized, desiccated, or dried form as appropriate, or may be provided in an aqueous solution or other liquid media appropriate for use in accordance with the disclosure.

Kits useful for the present disclosure may also include additional reagents, e.g., buffers, media components, such as salts including MgCl₂, a polymerase enzyme, primers, and deoxyribonucleotides, and the like, reagents for DNA isolation, or the like, as described herein. Such reagents or components are well known in the art. Where appropriate, reagents included with such a kit may be provided either in the same container or media as a primer pair or multiple primer pairs, or may alternatively be placed in a second or additional distinct container into which an additional composition or reagents may be placed and suitably aliquoted. Alternatively, reagents may be provided in a single container means. A kit of the disclosure may also include instructions for use, including storage requirements for individual components as appropriate.

### VII. Definitions

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms in molecular biology may also be found in Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al, A Dictionary of Genetics, 6th ed., Oxford University Press: New York, 2002; and Lewin, Genes IX, Oxford University Press: New York, 2007. The nomenclature for DNA bases as set forth at 37 CFR § 1.822 is used.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1

### Qualitative Detection of Chromosomes in Human Samples

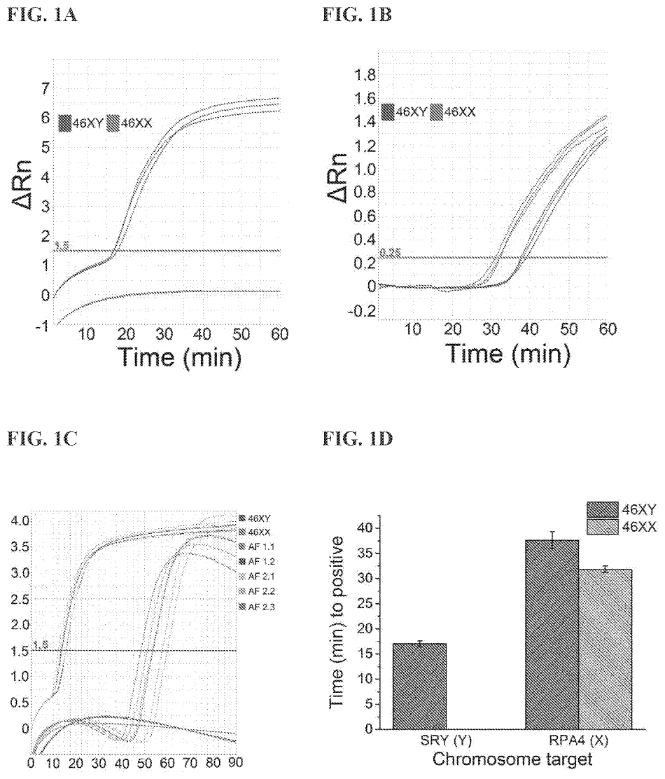

To determine whether LAMP could qualitatively detect target sequences in human whole-genome DNA samples, purified genomic DNA was isolated from peripheral blood of SNP microarray-confirmed normal male (46 XY) and female (46 XX) fibroblast cell lines, and amniotic fluid (AF) samples. LAMP using primer sets of six probes for two unique single-copy target genes with invariant loci specific for the Y chromosome (SRY) and X chromosome (RPA4), were performed LAMP using turbidity/absorbance- and fluorescence-based reporter assays and detection principles utilized and described previously. The highest quality, sensitivity, specificity and accuracy was obtained using target-specific fluorescent tags (probes) by real-time monitoring of fluorescence increase during product amplification (FIG. 1). Under these conditions, it was possible to readily distinguish between male and female sample with clear differentiation within only 15-30 min and requiring as little as 1-2 ng of genomic DNA (FIG. 1A, FIG. 1B, and FIG. 1D).

In case of original or treated clinical AF samples containing very low DNA concentrations (tens to several fetus genome copies in small tested sample aliquots) the sex discrimination was achieved at 45-60 min, depending on sample processing method (FIG. 1C).

Overall, these preliminary results indicate that each human DNA target may be rapidly and reliably qualitatively determined in presence/absence analysis of both control DNA and AF samples by means of LAMP-based reactions. While this could serve as a new efficient and convenient tool for rapid and precise embryo and neonatal sex determination, such as in cases of ambiguous genitalia, far more useful would be a quantitative method that could distinguish relative copy number for different target sequencing in a genomic DNA sample.

### Example 2

### Quantitative Target Detection: Isolated Target

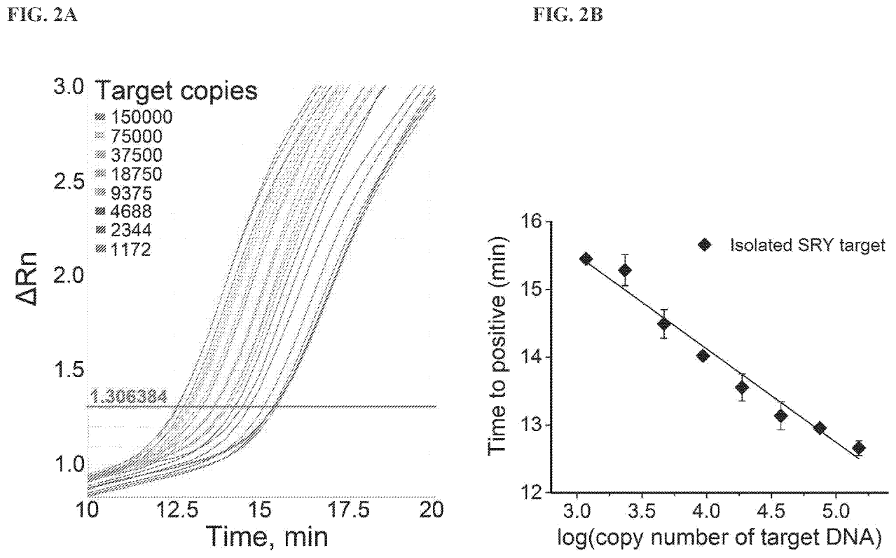

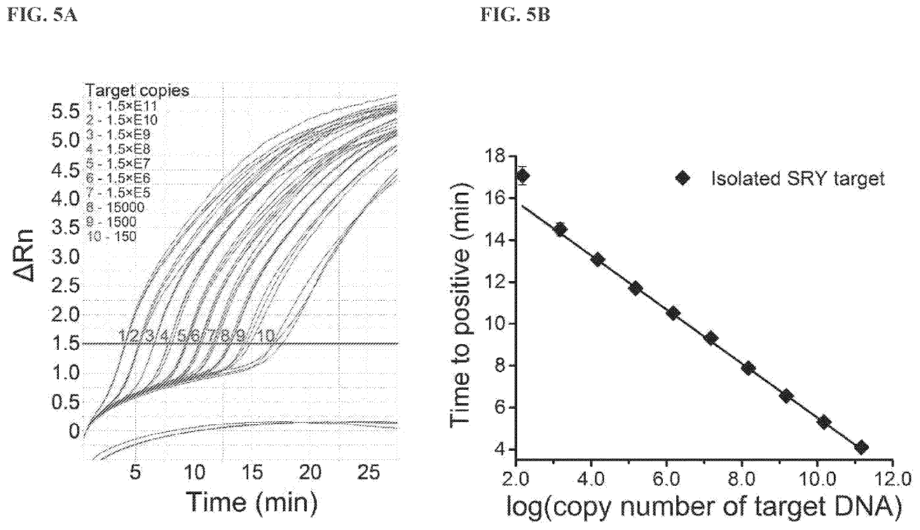

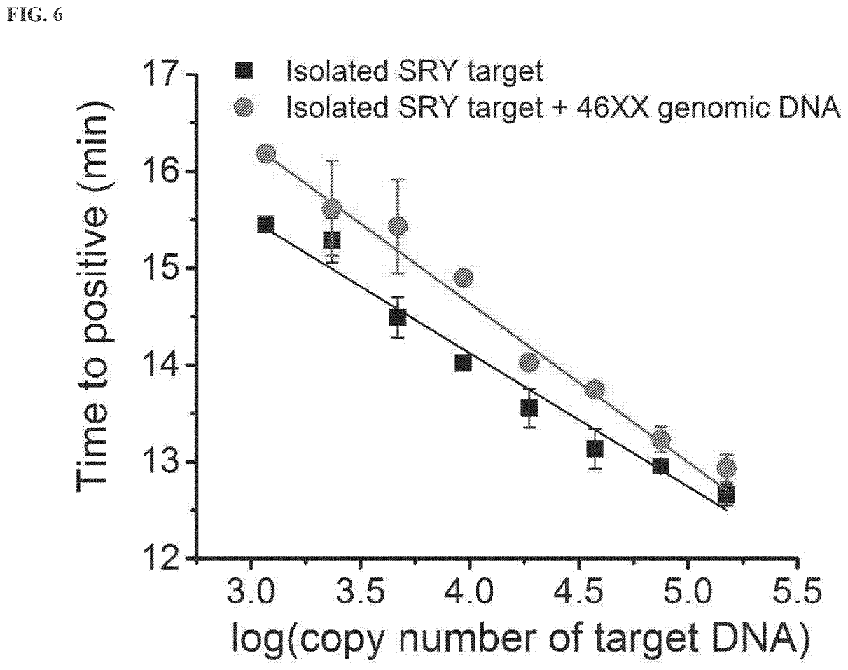

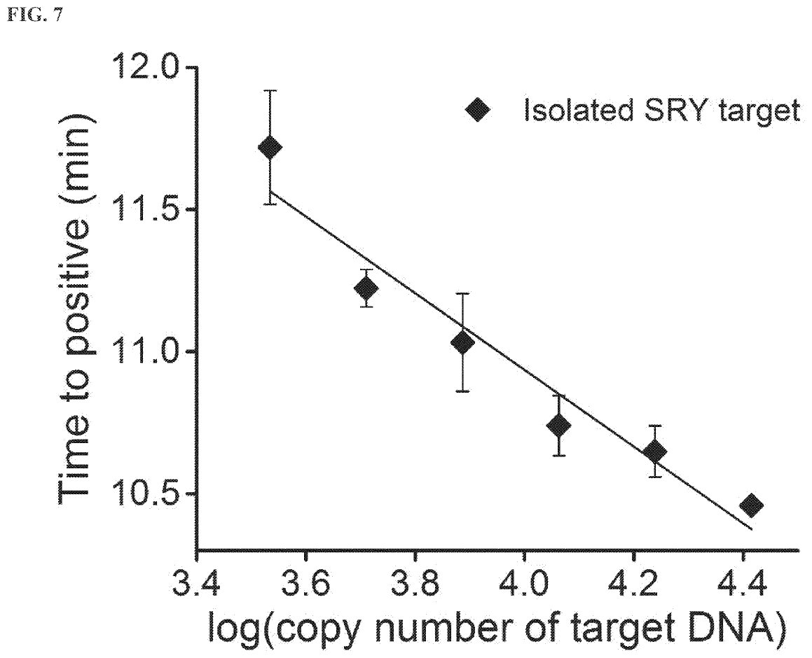

To develop a method for quantitative analysis of target DNA using qLAMP, the ability of qLAMP to detect purified small DNA targets of varying concentrations was tested. Two, unique, single-copy target sequences specific for human X and Y sex chromosomes (RPA4 region and SRY region, respectively) were pre-amplified from genomic DNA template and tested with qLAMP. Trisomies, such as trisomy 21 (aka Down Syndrome) represent a 1.5-fold increase in the number of the affected chromosome. Thus, to be an effective screening or diagnostic test, qLAMP would need to o detect a 1.5-fold difference in target DNA concentration. We prepared 10-fold (FIG. 5), 2-fold and 1.5-fold serial dilutions of isolated SRY target ranging from 1.5×1011 to 150 to copies. Across the full dilution range, qLAMP calibration curves for 10× dilutions were highly reproducible copies (FIG. 2A, FIG. 2B; FIG. 6). For the 2-fold and 1.5-fold dilutions, highly reproducible results were obtained within the 1.5×105-1172 (max SD=0.2278) and 2.6×104-3424 (SD=0.1999) SRY target copy range, respectively, (FIG. 7).

To determine whether the presence of genomic DNA would interfere with qLAMP, the serial dilutions were tested in the presence of an equimolar genome copy number of 46 XX genomic DNA, which lacks the SRY target sequence.

As shown in FIG. 6, the presence of 46 XX genomic DNA did not interfere with the amplification accuracy and efficiency and caused only a slightly delay in the amplification process. Overall, these data indicate that, at least at the level of isolated DNA, a 2- to 1.5-fold differences in target copy number may be easily and reliably resolved even with initial input as small as 1172 target copy number. The previously published observation that the presence of non-target DNA itself does not appear to impact on detection accuracy and precision was also confirmed.

### Example 3

### Quantitative Target Detection: Whole Genome Samples

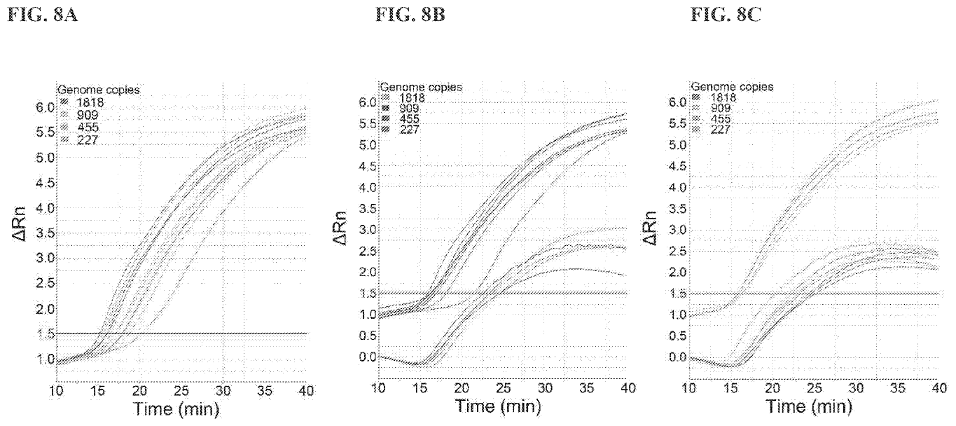

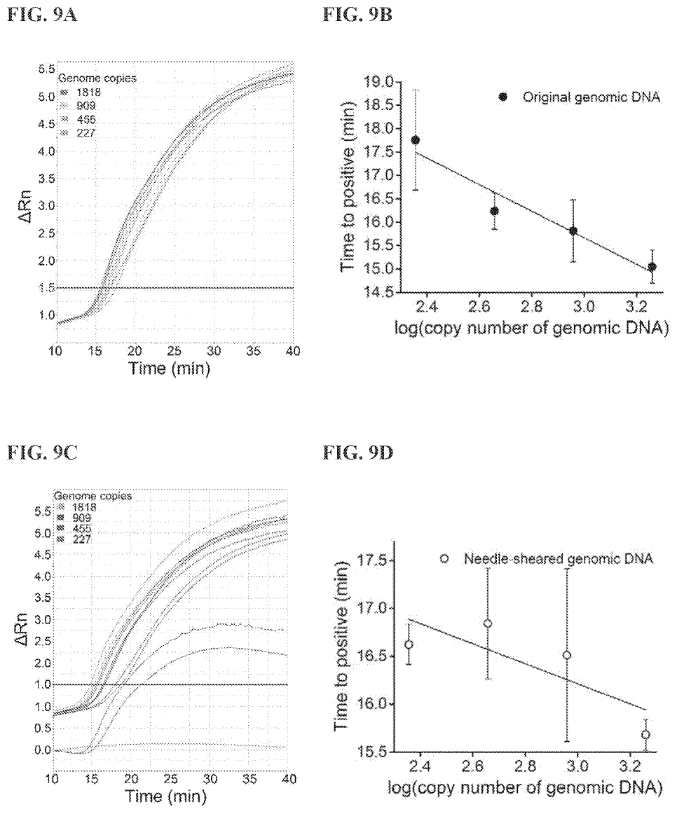

Next, the ability of LAMP to quantifiably measure concentration differences in target concentrations was tested within whole genome DNA isolated from peripheral blood samples and cell cultures. How target accessibility for isothermal amplification (LAMP) and quantification is altered based on DNA quality and fragmentation was first evaluated. Genomic DNA samples were treated with enzymatic digestion (non-site specific nucleases, such as fragmentase or micrococcal nuclease), mechanical digestion (shearing or sonication to partly digest DNA), and DNA pre-heating. Resultant samples were tested with qLAMP to optimize further steps of DNA sample processing. Enzymatic and mechanical digestion of target genomic DNA were shown to be insufficient and failed to improve qLAMP efficiency, accuracy precision to correctly quantify target sequences (FIG. 8 and FIG. 9). Depending on time and intensity of digestion, detected amplification rates and reproducibility within replicates were either insufficient to plot dilution curves and detect differences between serial dilutions of fragmented input DNA (FIG. 8) or, at least, were comparable to those for original (untreated) DNA (FIG. 9). Presumably, these treatment procedures caused DNA damage that impaired the ability of qLAMP to effectively amplify the target sequence.

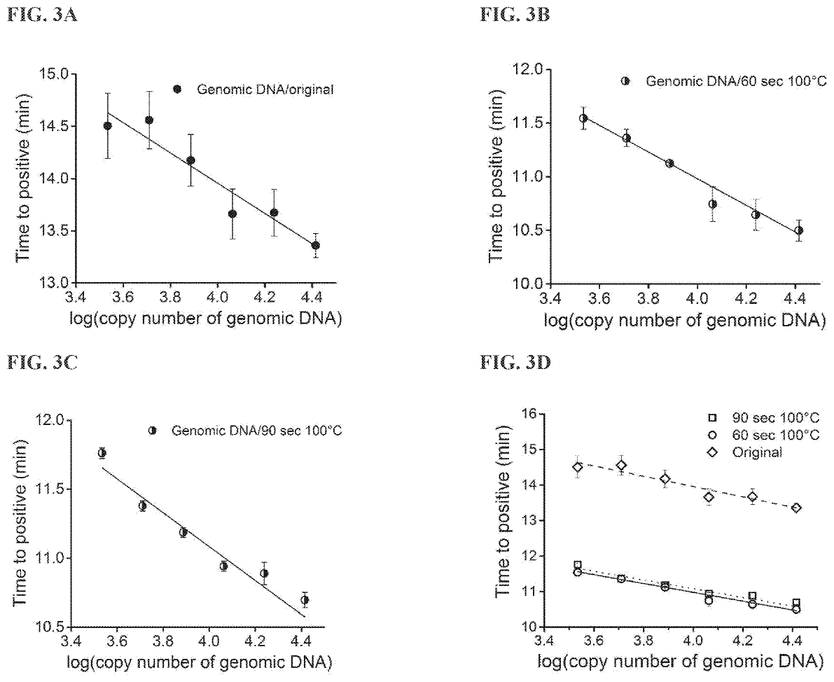

It was hypothesized that a brief heat denaturation step might remove tertiary DNA structure from human genomic DNA samples thereby improving target accessibility for LAMP and reducing variability between samples, thus increasing accuracy and precision of the assay. Genomic DNA samples were preheated to 100° C. for time intervals ranging from 10-180 sec compared their amplification rates to original genomic DNA and isolated targets. As shown in FIG. 3, pre-heating heating genomic DNA samples improved SRY target amplification efficiency, reproducibility, and accuracy (FIG. 3).

### Example 4

### Quantification of Sex Chromosomes

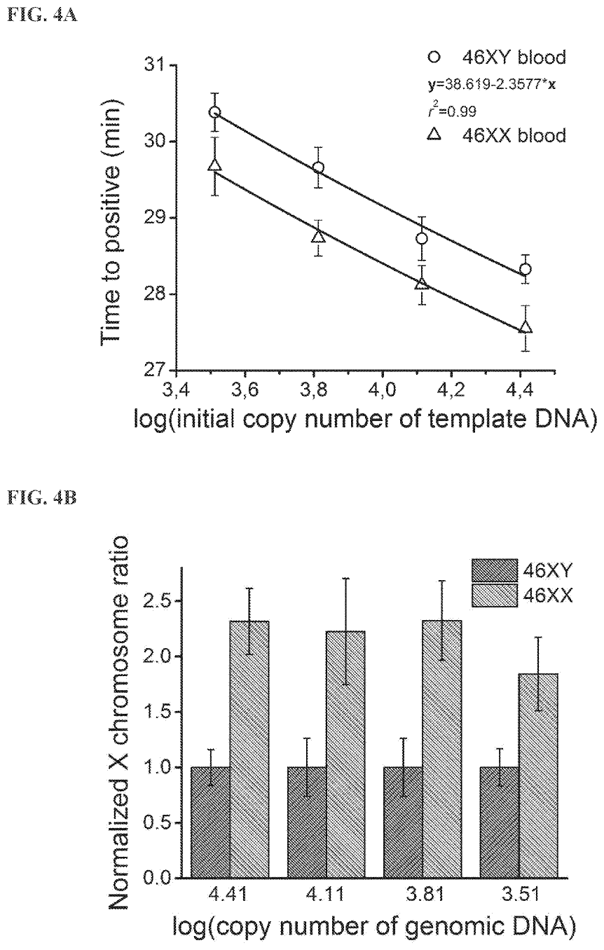

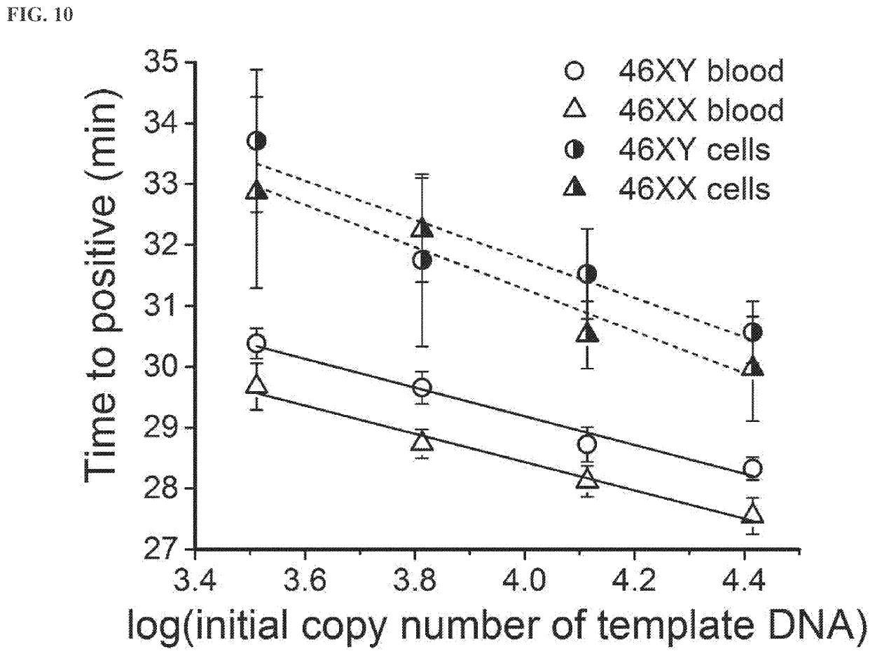

The ability of LAMP to measure relative X-chromosome content was next assessed in whole genome DNA samples of normal male and female karyotypes (46 XY, 46 XX) originated from blood cells and cell cultures. The aim was to reliably distinguish a 2-fold ratio of X chromosome copy number. Identical 2-fold serial dilutions were prepared in replicates ranging from 85.8 ng (26×10³ genome copies) to 10.7 ng (3250 genome copies) of each pre-heated DNA sample and tested with the LAMP-based assay to quantify relative RPA4 target copy number. Ct values obtained corresponding to amplification time to reach positive signal were used to plot dilution curves (FIG. 4A). The 46 XY sample was used as the reference sample for signal normalization. RPA4 target copy number in 46 XX sample at each dilution point was calculated according to the equation describing the dilution (standard) curve for reference 46 XY (FIG. 4A). Calculated number of RPA4-targets (i.e., X chromosome) copies detected in 46 XX sample were normalized to reference copy number in a 46 XY sample at respective dilution points. Standard deviations were calculated for triplicates of each dilution point (intra-assay) and also normalized to reference sample. The test was repeated and reproduced in two independent experiments (inter-assay); inter-assay standard deviations were also calculated (FIG. 4A and FIG. 4B). Thus, LAMP can measure the expected 1:2 relative quantification of RPA4 necessary to distinguish genomic DNA samples of 46 XY and 46 XX karyotypes using pre-heated genomic DNA samples isolated from peripheral blood samples. In contrast, DNA originated from cultured cell lines even after pre-heating steps showed poor quality for LAMP to access and quantify RPA4 target correctly (FIG. 10). Replicate signal deviations at each dilution point were high dramatically decreasing the accuracy of analysis.

Collectively, the data demonstrate an advantageous ability of LAMP to qualitatively detect target sequences in human genomic DNA samples extracted from peripheral blood, tissue and amniotic fluid and, more importantly, quantitatively measuring relative levels of target sequences, e.g., chromosome copy numbers.

### Example 5

### Optimization of LAMP

Quantitative 2-plex LAMP capable of detecting 1:2 difference was performed as described below. In particular, the enzyme concentration used in LAMP experiments was increased, the Tm was increased, and the primer concentration was reduced. In addition, the Mg2+ concentration was reduced for droplet assay experiments, resulting in a more robust and specific reaction than quantitative LAMP experiments with ultra-low DNA input <0.1 ng).

Two-plex LAMP was also performed for aneuploidy detection with lower DNA input. For example, a DNA panel (46 XX, 46 XY, 45 XO, 47, XXX) was used at different concentrations, including 60 ng, 30 ng, 10 ng, and 3 ng.

Detection sensitivity and power were improved by introducing more technical repeats, i.e., 3 vs 10 repeats, as well as additional primer pairs.

### Example 6

### Chromosome-Specific Targets and LAMP Primers Design

Further testing was performed for aneuploidy detection using isothermic PCR. Two unique single-copy target genes with invariant loci were identified and selected for each of human sex chromosomes through mining both literature (15) and databases (International HapMap Project, Entrez SNP, DECIPHER (Database of Chromosomal Imbalance and Phenotype in Humans using Ensembl Resources, Decipher Consortium)) (16,17): SRY (sex determining region Y [ID 6736, NCBI]) specific for the Y chromosome and RPA4 (replication protein A4 [ID29935, NCBI]) specific for the X chromosome. LAMP primer sets of six primers (FIP, BIP, F3, B3, LF, LB) per each target were designed with the aid of LAMP primer designing software PrimerExplorer V4 (SI, Table 1). LAMP primers for CFTR (cystic fibrosis transmembrane conductance regulator [ID 1080, NCBI]) gene target used as internal reference control for signal normalization were published elsewhere (18).

Additional probes represented by fluorescently labeled FIP primer aligned together with complementary Fd oligo linked to quencher were designed and synthesized to ensure target-specific fluorescent-based read-out to detect and discriminate each unique target in singleplex/multiplex assays. Primer sequences are provided in Table 2.

### Example 7

### Target DNA Samples

For initial development of qLAMP, a 200 bp PCR product from the human SRY gene (sex determining region Y [ID 6736, NCBI]) and RPA4 gene (replication protein A4 [ID29935, NCBI]) was purified using QIAquick PCR purification kit (QIAGEN) followed by confirmation by Sanger was used as a target sequence. Primer sequences used are shown in Supplementary Table 2. Serial dilutions of the purified PCR fragments were tested first to optimize the LAMP parameters for correct quantification and to characterize test limitations.

Subsequent testing on human genomic samples utilized DNA samples isolated from primary blood cells of adults and well-characterized lymphoblastoid cell lines from B-lymphocytes with normal male (46 XY) [GM12877, Coriell] and female (46 XX) [GM12878 Coriell] karyotypes. Samples with abnormal ratios of the sex chromosomes (47 XYY, 45 XO) originated from respective cell line of Coriell Cell Repository (e.g., fibroblast cell line, XYY syndrome [GM11337]) and from original human tissue samples. Intact whole genome DNA was isolated from tissues and blood or cultured cells by means of appropriate column-based manufacture protocols (QIAamp® DNA Mini kit, QIAamp® DNA Blood Mini/Midi kits, QIAGEN). Amniotic fluid (AF) samples were collected from pregnant patients and genomic DNA was isolated using QIAamp® DNA Mini kit (QIAGEN). Genomic DNA was isolated based on different protocols including freezing (−80° C.) and thawing (37° C.) cycles, adding equal volumes of lysis buffer, and sonication (3-10 sec at stage 1-2 intensity level (Microson™ XL2000, Qsonica, LLC)).

To release secondary structure from genomic DNA, samples were incubated at 100° C. for various times ranging from 30-120 sec. Both untreated intact genome DNA (control) and pre-heated DNA were used to prepare 10-, 2- and 1.5-fold serial dilutions and test them with LAMP for specific target quantification.

### Example 8

### LAMP Reaction

LAMP was performed using target-specific fluorescent probes (e.g., 5′-Quencher-FIP:Fd-Fluorophore-3′ (Q-FIP:Fd-Fluo) duplex), prepared immediately prior to use by mixing equal volumes of 200 μM 5′-Quencher-FIP (5′-modification) and 200 μM Fd-Fluorophore (3′-modification) and double volume of nuclease-free water to reach 50 μM concentration of each oligonucleotide (18). The mixture was heated for 3 min at 98° C. to align complementary sequences, followed by slow gradual cooling to room temperature.

Complete LAMP reaction with target-specific fluorescent probes contained 0.8 μM FIP and 0.4 μM Q-FIP:Fd-Fluo duplex, 1.6 μM BIP, 0.2 μM each of F3 and B3, 0.4 μM each of LF and LB; 2.8 U Bst 2.0 WarmStart DNA polymerase (New England Biolabs (NEB)); 1 mM each of dNTPs (NEB); DNA template (tested dilution); 1× Isothermal Amplification Buffer additionally supplemented with 6 mM MgSO₄ (NEB), 1×ROX reference dye (0.5 μM). For multiplex reactions, total primer concentrations were kept to those described for the standard LAMP reaction, but with each set representing 1/n of the total, where n is the number of targets and respective primer sets.

LAMP reactions were performed in 10 μl volumes. All tested DNA samples were analyzed in duplicates or triplicates within an experiment (intraassay) and in independent experiments (interassay). The reaction plate was incubated at 62° C. for 60 min and monitored in real-time mode using qPCR real-time cycler (StepOnePlus™ Real-Time PCR Systems, Applied Biosystems) with 30-sec cycling steps.

### Example 9

### Data Analysis, Statistics

Ct values, reflecting time to reach positive signal threshold, were used to plot calibration curves as a function of target copy number input load for each separate target in the reference sample (46 XY karyotype). Respective amplification Ct values for each dilution of test samples were used to calculate input target copy number in those samples. Obtained target copy number in each dilution of test sample was normalized to the respective value for reference sample dilution to calculate target copy number ratio.

Means and variances of the rates of concurrence was evaluated for significance with Student's t-test with determination of effect size along with p-values and standard deviations within each experiment for duplicates and triplicates (intra-assay) and between independent experiments (inter-assay).

### Example 10

### Results—Qualitative Detection of Chromosomes in Human Samples

To determine whether LAMP could qualitatively detect target sequences in human whole-genome DNA samples, purified genomic DNA was isolated from peripheral blood of SNP microarray-confirmed normal male (46 XY) and female (46 XX), fibroblast cell lines, and amniotic fluid (AF) samples. LAMP using primer sets of six probes for two unique single-copy target genes with invariant loci specific for the Y chromosome (SRY) and X chromosome (RPA4), were performed LAMP using turbidity/absorbance- and fluorescence-based reporter assays and detection principles utilized and described previously (19). The highest quality, sensitivity, specificity and accuracy was obtained using target-specific fluorescent tags (probes) by real-time monitoring of fluorescence increase during product amplification (FIG. 1) (18). Under these conditions, it was possible to distinguish between male and female samples with clear differentiation within only 15-30 min and requiring as little as 1-2 ng of genomic DNA (FIG. 1A, FIG. 1B, FIG. 1D).

In case of original or treated clinical AF samples containing very low DNA concentrations (tens to several fetus genome copies in small tested sample aliquots) the sex discrimination was achieved at 45-60 min, depending on sample processing method (FIG. 1C).

Overall, these preliminary results indicate that each human DNA target may be rapidly and reliably qualitatively determined in presence/absence analysis of both control DNA and AF samples by means of LAMP-based reactions. While this could serve as a new efficient and convenient tool for rapid and precise embryo and neonatal sex determination, such as in cases of ambiguous genitalia, far more useful would be a quantitative method that could distinguish relative copy number for different target sequencing in a genomic DNA sample.

### Example 11

### Results—Quantitative Target Detection: Isolated Target

To develop a method for quantitative analysis of target DNA using qLAMP, the ability of qLAMP to detect purified small DNA targets of varying concentrations was first tested. Two unique, single-copy target sequences specific for human X and Y sex chromosomes (RPA4 region and SRY region, respectively) were pre-amplified from genomic DNA template and tested with qLAMP. Trisomies, such as trisomy 21 (aka Down's Syndrome) represent a 1.5-fold increase in the number of the affected chromosome. Thus, to be an effective screening or diagnostic test, qLAMP would need to detect a 1.5-fold difference in target DNA concentration. We prepared 10-fold (FIG. 5), 2-fold and 1.5-fold serial dilutions of isolated SRY target ranging from 1.5×10¹¹ to 150 copies. Across the full dilution range, qLAMP calibration curves for 10× dilutions were highly reproducible copies (from about 1500 to about 1.5×10¹¹ copies) (FIG. 2A, FIG. 2B; FIG. 6). For the 2-fold and 1.5-fold dilutions, highly reproducible results were obtained within the 1.5×10⁵-1172 (max SD=0.2278) and 2.6×10⁴-3424 (SD=0.1999) SRY target copy range, respectively, (FIG. 7).

To determine whether the presence of genomic DNA would interfere with qLAMP, the serial dilutions were tested in the presence of an equimolar genome copy number of 46 XX genomic DNA, which lacks the SRY target sequence.

The presence of 46 XX genomic DNA did not interfere with the amplification accuracy and efficiency and caused only a slightly delay in the amplification process (FIG. 6). Overall, these data indicate that, at least at the level of isolated DNA, a 2- to 1.5-fold differences in target copy number may be easily and reliably resolved even with initial input as small as 1172 target copy number. It was also confirmed that the presence of non-target DNA itself does not appear to impact on detection accuracy and precision.

### Example 12

### Results—Quantitative Target Detection: Whole Genome Samples

Next, we tested the ability of LAMP to quantifiably measure concentration differences in target concentrations within whole genome DNA isolated from peripheral blood samples and cell cultures. We first evaluated how target accessibility for isothermal amplification (LAMP) and quantification is altered based on DNA quality and fragmentation. Genomic DNA samples were treated with enzymatic digestion (non-site-specific nucleases, such as fragmentase or micrococcal nuclease), mechanical digestion (shearing or sonication to partly digest DNA), and DNA pre-heating. Resultant samples were tested with qLAMP to optimize further steps of DNA sample processing. Enzymatic and mechanical digestion of target genomic DNA were shown to be insufficient and failed to improve qLAMP efficiency, accuracy precision to correctly quantify target sequences (FIG. 8 and FIG. 9). Depending on time and intensity of digestion, detected amplification rates and reproducibility within replicates were either insufficient to plot dilution curves and detect differences between serial dilutions of fragmented input DNA (FIG. 8) or, at least, were comparable to those for original (untreated) DNA (FIG. 9). Presumably, these treatment procedures caused DNA damage that impaired the ability of qLAMP to effectively amplify the target sequence.

We hypothesized that a brief heat denaturation step might remove tertiary DNA structure from human genomic DNA samples thereby improving target accessibility for LAMP and reducing variability between samples, thus increasing accuracy and precision of the assay. We pre-heated genomic DNA samples to 100° C. for time intervals ranging from 10-180 sec compared their amplification rates to original genomic DNA and isolated targets. As shown in FIG. 3, pre-heating genomic DNA samples improved SRY target amplification efficiency, reproducibility, and accuracy.

### Example 13

### Results—Quantification of Sex Chromosomes

We next assessed the ability of LAMP to measure relative X-chromosome content in whole genome DNA samples of normal male and female karyotypes (46 XY, 46 XX) originated from blood cells and cell cultures. We aimed to reliably distinguish a 2-fold ratio of X chromosome copy number. We prepared identical 2-fold serial dilutions (in replicates) ranging from 85.8 ng (26×103 genome copies) to 10.7 ng (3250 genome copies) of each pre-heated DNA sample and tested them with the LAMP-based assay to quantify relative RPA4 target copy number. Ct values obtained corresponding to amplification time to reach positive signal were used to plot dilution curves (FIG. 4A). The 46 XY sample was used as the reference sample for signal normalization. RPA4 target copy number in 46 XX sample at each dilution point was calculated according to the equation describing the dilution (standard) curve for reference 46 XY (FIG. 4A). Calculated number of RPA4-targets (i.e., X chromosome) copies detected in 46 XX sample were normalized to reference copy number in a 46 XY sample at respective dilution points. Standard deviations were calculated for triplicates of each dilution point (intra-assay) and also normalized to reference sample. The test was repeated and reproduced in two independent experiments (inter-assay); interas say standard deviations were also calculated (FIG. 4A, FIG. 4B). Thus, LAMP can measure the expected 1:2 relative quantification of RPA4 necessary to distinguish genomic DNA samples of 46 XY and 46 XX karyotypes using pre-heated genomic DNA samples isolated from peripheral blood samples. In contrast, DNA originated from cultured cell lines even after pre-heating steps showed poor quality for LAMP to access and quantify RPA4 target correctly (FIG. 10). Replicate signal deviations at each dilution point were high dramatically decreasing the accuracy of analysis.

Collectively, the data demonstrate an advantageous ability of LAMP to qualitatively detect target sequences in human genomic DNA samples extracted from peripheral blood, tissue and amniotic fluid and, more importantly, quantitatively measuring relative levels of target sequences, e.g., chromosome copy numbers.

### Example 14

### Discussion

The results demonstrate that qLAMP-based technique may be successfully applied for not only qualitative target detection in human genomic DNA samples, but also for accurate and precise target relative quantification with high resolution. This can provide correct relative quantification of chromosome copy number ratio or CNVs.

In contrast, DNA originated from cultured cell lines even after pre-heating steps showed poor quality for LAMP to access and quantify RPA4 target correctly (FIG. 10). Replicate signal deviations at each dilution point were high dramatically decreasing the accuracy of analysis. Even when attempting to quantify relative chromosome copy number differences in DNA samples (untreated or treated in universal manner, e.g., DNA short-term pre-heating) originated from different sources (e.g., 46 XY and 46 XX DNA from blood cells and cultured cells vs. 47 XYY DNA from cultured cells vs. 45 XO DNA from frozen tissue) upon normalization to an internal reference (e.g., CFTR gene), the variability/deviations of obtained results were large enough for clinically meaningful use. Primary mature cells and cultured dividing and growing cells undergo different functional and developmental processes, thus genomic DNA may have specific confirmation, configuration and modifications. For example, adult primary cells (e.g., blood cells) are all universal in terms of constant developmental level, while growing cultured cell populations are heterogeneous as a result of de-synchronized cells cycles thus appearing different DNA state. We hypothesize that large genome DNA originated from different sources (mature blood cells, cultured cell lines, complex tissues heterogeneous in structure and content) may undergo different confirmation changes and modifications and this may be crucial for accuracy and precision of highly selective and target specific LAMP-based quantification. Nevertheless, we suppose that DNA originated from type-specific primary source (e.g. type-specific primary cells, such as blood cells) will be universal and homogenous in terms of confirmation/modification state and simple DNA treatment such as pre-heating should be suitable to process DNA for quantitative LAMP.

It may be possible to use qLAMP for testing clinical samples for human genetic screening purposes, e.g., aneuploidy and CNV detection in prenatal diagnosis and PGS.

### Example 15

### Primers Used for Multiplex qLAMP

TABLE 3
Primers used for multiplex qLAMP in
addition to RPA4 and SRY primers.

| GJBP5 (Chr 1) | 5′-seq-3′ | modification | |
|---|---|---|---|
| 1_GJB5-FIP | ATCACTCCACACACGCTCGGCT | | |
| | CTGGTCTTCATCTTCCGC | | |
| | (SEQ ID NO: 26) | | |
| 1_GJBP5-BIP | CGACTGCAATACTCGCCAGCCT | | |
| | GGGACACAGGGAAGAACTC | | |
| | (SEQ ID NO: 27) | | |
| 1_GJBP5-F3 | TCAACAAGTACTCCACAGCC | | |
| | (SEQ ID NO: 28) | | |
| 1_GJBP5-B3 | GCATGTCACCAGGATAAGCT | | |
| | (SEQ ID NO: 29) | | |
| 1_GJBP5-LF | CGTCACCAGGTACACCAGCA | | |
| | (SEQ ID NO: 30) | | |
| 1_GJBP5-LB | TGCTCCAACGTCTGCTTTG | | |
| | (SEQ ID NO: 31) | | |
| 1_GJBP5.Flc | ATCACTCCACACACGCTCGG | | |
| | (SEQ ID NO: 32) | | |
| 1_GJBP5-F2 | GCTCTGGTCTTCATCTTCCGC | | |
| | (SEQ ID NO: 33) | | |
| 1_GJBP5-5IABkFQ- | /5IABkFQ/ATCACTCCACACAC | 5′ | Iowa |
| FIP | GCTCGGCTCTGGTCTTCATCTTC | | Black |
| | CGC | | ® FQ |
| | (SEQ ID NO: 34) | | |
| 1_GJBP5-3JOE_N- | CCGAGCGTGTGTGGAGTGA | 3′ | JOE (NHS |
| Fd | T/3JOE_N/ | | ester) |
| | (SEQ ID NO: 35) | | |
| MEF2D.e5 (Chr 1) | | | |
| MEF2D.e5.F3 | GATGATGTCACCAGGGAAGG | | |
| | (SEQ ID NO: 36) | | |
| MEF2D.e5.B3 | CCTCCCAGGAAAAGTGGACT | | |
| | (SEQ ID NO: 37) | | |
| MEF2D.e5.FIP | TTGCCATGCCTGTCACGGTG-CCCC | | |
| | TGGGATTGCTGAAC | | |
| | (SEQ ID NO: 38) | | |
| MEF2D.e5.BIP | GGCCGGGACAGTTGACTAGAC-CCA | | |
| | TGGAAGGGGTCAACCT | | |
| | (SEQ ID NO: 39) | | |
| MEF2D.e5.LF | GTCCAATCAGAGCTCACTGCA | | |
| | (SEQ ID NO: 40) | | |
| MEF2D.e5.LB | GAAAGATGGAGGGGCAGGATCAG | | |
| | (SEQ ID NO: 41) | | |
| MEF2D.e5.5IABkFQ- | /5IABkFQ/TTGCCATGCCTGTCAC | 5′ | Iowa |
| FIP | GGTG-CCGCTGGGATTGCTGAAC | | Black |
| | (SEQ ID NO: 42) | | ® FQ |
| MEF2D.e5.3JOE_N- | CACCGTGACAGGCATGGCA | 3′ | JOE |
| Fd | A/3JOE_N/ | | |
| | (SEQ ID NO: 43) | | |
| PHAK2.C8 (Chr X) | | | |
| PHAK2.e8.F3 | GGGGTTCAAAGGTGCCTG | | |
| | (SEQ ID NO: 44) | | |
| PHAK2.e8.B3 | CCTGTGTGATGTGCATGGAG | | |
| | (SEQ ID NO: 45) | | |
| PHAK2.e8.FIP | AGGCAAGAAAACAAAGTCTCCGAG | | |
| | T-TGAGTGCCAGTTCTGCTTC | | |
| | (SEQ ID NO: 46) | | |
| PHAK2.e8.BIP | AGACTCAGCTTTGAACGCTGTATG | | |
| | C-AGCTCACCCATGCCTCAG | | |
| | (SEQ ID NO: 47) | | |
| PHAK2.e8.LF | TTAGTGGCTTGGTTCTCTCCTTGA | | |
| | (SEQ ID NO: 48) | | |
| PHAK2.e8.LB | AATCAAGCAATTAACCTCATGGGGA | | |
| | (SEQ ID NO: 49) | | |
| PHAK2.e8.5IABkFQ- | /5IABkFQ/AGGCAAGAAAACAAA | 5′ | Iowa |
| FIP | GTCTCCGAGT-TGAGTGCCAGTTCT | | Black® |
| | GCTTC | | FQ |
| | (SEQ ID NO: 50) | | |
| PHAK2.e8.36FAM- | ACTCGGAGACTTTGTTTTCTTGC | 3′ | 6-FAM |
| Fd | CT//36-FAM/ | | |
| | (SEQ ID NO: 51) | | |
| UTY.e1 (ChrY) | | | |
| UTY.e1.F3 | CTTTAGGAGAGTCCGTAATGAGG | | |
| | (SEQ ID NO: 52) | | |
| UTY.e1.B3 | GGCTTACCGACTGAGGTCAT | | |
| | (SEQ ID NO: 53) | | |
| UTY.e1.FIP | AAGGTTTAGGGCCTGCGCAG-TC | | |
| | CCACTGTCACAAGCCT | | |
| | (SEQ ID NO: 54) | | |
| UTY.e1.BIP | CTTTTGGCCCTAAGGCCTTGTC | | |
| | A-GCCTTCTCTTTAGACTTGGT | | |
| | CA | | |
| | (SEQ ID NO: 55) | | |
| UTY.e1.LF | TGCCATGCCCCGCAAG | | |
| | (SEQ ID NO: 56) | | |
| UTY.e1.LB | CCTCCATAGGCTGGTTCTTTGC | | |
| | (SEQ ID NO: 57) | | |
| UTY.e1.F1c | AAGGTTTAGGGCCTGCGCAG | | |
| | (SEQ ID NO: 58) | | |
| UTY.e1.5IABkFQ- | /5IABkFQ/AAGGTTTAGGGCCT | 5′ | Iowa |
| FIP | GCGCAG-TCCCACTGTCACAAGC | | Black® |
| | CT | | FQ |
| | (SEQ ID NO: 59) | | |
| UTY.e1.36FAM-Fd | CTGCGCAGGCCCTAAACCTT/ | 3′ | 6-FAM |
| | 36-FAM/ | | |
| | (SEQ ID NO: 60) | | |

### REFERENCES

1. Lee, H. L., McCulloh, D. H., Hodes-Wertz, B., Adler, A., McCaffrey, C. and Grifo, J. A. (2015) In vitro fertilization with preimplantation genetic screening improves implantation and live birth in women age 40 through 43. Journal of assisted reproduction and genetics, 32, 435-444.
2. Meberg, A., Hals, J. and Thaulow, E. (2007) Congenital heart defects-chromosomal anomalies, syndromes and extracardiac malformations. Acta Paediatr, 96, 1142-1145.
3. Rosenfeld, J. A., Tucker, M. E., Escobar, L. F., Neill, N. J., Torchia, B. S., McDaniel, L. D., Schultz, R. A., Chong, K. and Chitayat, D. (2015) Diagnostic utility of microarray testing in pregnancy loss. Ultrasound in obstetrics & gynecology: the official journal of the International Society of Ultrasound in Obstetrics and Gynecology, 46, 478-486.
4. Baker, K., Sanchez-de-Toledo, J., Munoz, R., Orr, R., Kiray, S., Shiderly, D., Clemens, M., Wearden, P., Morell, V. O. and Chrysostomou, C. (2012) Critical congenital heart disease—utility of routine screening for chromosomal and other extracardiac malformations. Congenital heart disease, 7, 145-150.
5. Charan, P., Woodrow, N., Walker, S. P., Ganesamoorthy, D., McGillivray, G. and Palma-Dias, R. (2014) High-resolution microarray in the assessment of fetal anomalies detected by ultrasound. The Australian & New Zealand journal of obstetrics & gynaecology, 54, 46-52.
6. Leclercq, S., Lebbar, A., Grange, G., Tsatsaris, V., Le Tessier, D. and Dupont, J. M. (2008) Optimized criteria for using fluorescence in situ hybridization in the prenatal diagnosis of common aneuploidies. Prenatal diagnosis, 28, 313-318.
7. Miller, D. T., Adam, M. P., Aradhya, S., Biesecker, L. G., Brothman, A. R., Carter, N. P., Church, D. M., Crolla, J. A., Eichler, E. E., Epstein, C. J. et al. (2010) Consensus statement: chromosomal microarray is a first-tier clinical diagnostic test for individuals with developmental disabilities or congenital anomalies. American journal of human genetics, 86, 749-764.
8. Van Opstal, D., Boter, M., de Jong, D., van den Berg, C., Bruggenwirth, H. T., Wildschut, H. I., de Klein, A. and Galjaard, R. J. (2009) Rapid aneuploidy detection with multiplex ligation-dependent probe amplification: a prospective study of 4000 amniotic fluid samples. European journal of human genetics:EJHG, 17, 112-121.
9. Notomi, T., Okayama, H., Masubuchi, H., Yonekawa, T., Watanabe, K., Amino, N. and Hase, T. (2000) Loop-mediated isothermal amplification of DNA. Nucleic acids research, 28, E63.
10. Notomi, T., Mori, Y., Tomita, N. and Kanda, H. (2015) Loop-mediated isothermal amplification (LAMP): principle, features, and future prospects. Journal of microbiology, 53, 1-5.
11. Tomita, N., Mori, Y., Kanda, H. and Notomi, T. (2008) Loop-mediated isothermal amplification (LAMP) of gene sequences and simple visual detection of products. Nature protocols, 3, 877-882.
12. Fu, S., Qu, G., Guo, S., Ma, L., Zhang, N., Zhang, S., Gao, S. and Shen, Z. (2011) Applications of loop-mediated isothermal DNA amplification. Applied biochemistry and biotechnology, 163, 845-850.
13. Hirayama, H., Kageyama, S., Moriyasu, S., Sawai, K. and Minamihashi, A. (2013) Embryo sexing and sex chromosomal chimerism analysis by loop-mediated isothermal amplification in cattle and water buffaloes. The Journal of reproduction and development, 59, 321-326.
14. Nogami, H., Tsutsumi, H., Komuro, T. and Mukoyama, R. (2008) Rapid and simple sex determination method from dental pulp by loop-mediated isothermal amplification. Forensic science international. Genetics, 2, 349-353.
15. Scott, R. T. and Treff, N. R. (2010) US.
16. Firth, H. V., Richards, S. M., Bevan, A. P., Clayton, S., Corpas, M., Rajan, D., Van Vooren, S., Moreau, Y., Pettett, R. M. and Carter, N. P. (2009) DECIPHER: Database of Chromosomal Imbalance and Phenotype in Humans Using Ensembl Resources. American journal of human genetics, 84, 524-533.
17. International HapMap, C., Frazer, K. A., Ballinger, D. G., Cox, D. R., Hinds, D. A., Stuve, L. L., Gibbs, R. A., Belmont, J. W., Boudreau, A., Hardenbol, P. et al. (2007) A second generation human haplotype map of over 3.1 million SNPs. Nature, 449, 851-861.
18. Tanner, N. A., Zhang, Y. and Evans, T. C., Jr. (2012) Simultaneous multiple target detection in real-time loop-mediated isothermal amplification. BioTechniques, 53, 81-89.
19. Zhang, X., Lowe, S. B. and Gooding, J. J. (2014) Brief review of monitoring methods for loop-mediated isothermal amplification (LAMP). Biosensors & bioelectronics, 61, 491-499.
20. Nagamine, K., Watanabe, K., Ohtsuka, K., Hase, T. and Notomi, T. (2001) Loop-mediated isothermal amplification reaction using a nondenatured template. Clin Chem, 47, 1742-1743.
21. Njiru, Z. K. (2012) Loop-mediated isothermal amplification technology: towards point of care diagnostics. PLoS Negl Trop Dis, 6, e1572.
22. Brezina, P. R. and Kutteh, W. H. (2015) Clinical applications of preimplantation genetic testing. Bmj, 350, g7611.
23. De Sanctis, L. and Giuffre, M. (2010) Laboratory investigations in genetic syndromes: examples of clinical approach in the neonatal unit. Minerva pediatrica, 62, 193-195.
24. McDonald-McGinn, D. M. and Zackai, E. H. (2008) Genetic counseling for the 22q11.2 deletion. Dev Disabil Res Rev, 14, 69-74.
25. Bartlett, L. A., Berg, C. J., Shulman, H. B., Zane, S. B., Green, C. A., Whitehead, S. and Atrash, H. K. (2004) Risk factors for legal induced abortion-related mortality in the United States. Obstet Gynecol, 103, 729-737.
26. Christianson, A., Howson, C. P. and Modell, B. (2006), March of Dimes Birth Defects Foundation, White Plains, USA.
27. Bardos, J., Hercz, D., Friedenthal, J., Missmer, S. A. and Williams, Z. (2015) A national survey on public perceptions of miscarriage. Obstet Gynecol, 125, 1313-1320.
28. Foyouzi, N., Cedars, M. I. and Huddleston, H. G. (2012) Cost-effectiveness of cytogenetic evaluation of products of conception in the patient with a second pregnancy loss. Fertil Steril, 98, 151-155.
29. Hirayama, H., Kageyama, S., Takahashi, Y., Moriyasu, S., Sawai, K., Onoe, S., Watanabe, K., Kojiya, S., Notomi, T. and Minamihashi, A. (2006) Rapid sexing of water buffalo (Bubalus bubalis) embryos using loop-mediated isothermal amplification. Theriogenology, 66, 1249-1256.
30. Aoi, Y., Hosogai, M. and Tsuneda, S. (2006) Real-time quantitative LAMP (loop-mediated isothermal amplification of DNA) as a simple method for monitoring ammonia-oxidizing bacteria. J Biotechnol, 125, 484-491.
31. Cai, T., Lou, G., Yang, J., Xu, D. and Meng, Z. (2008) Development and evaluation of real-time loop-mediated isothermal amplification for hepatitis B virus DNA quantification: a new tool for HBV management. J Clin Virol, 41, 270-276.
32. Mori, Y., Kitao, M., Tomita, N. and Notomi, T. (2004) Real-time turbidimetry of LAMP reaction for quantifying template DNA. J Biochem Biophys Methods, 59, 145-157.

1. A method of analyzing one or more nucleic acid templates obtained from amniotic fluid samples, comprising:
a) providing a multiplex reaction mixture comprising:
at least one nucleic acid template to be amplified;
at least one nucleic acid target;
primers for amplifying the nucleic acid template; and
primers for amplifying the nucleic acid target;
b) co-amplifying the nucleic acid template and the nucleic acid target with a polymerase, wherein the co-amplification takes place isothermally, wherein the at least one nucleic acid template and the at least one nucleic acid target are heated to at least 94° C. prior to amplification, and wherein the at least one nucleic acid template and the at least one nucleic acid target are amplified;
c) normalizing the at least one nucleic acid template, wherein the normalization is carried out after completion of the co-amplification of step b); and
d) analyzing the one or more nucleic acid templates by testing for one or more of aneuploidy, copy number variations, genetic sex, and pathogens.

2. The method of claim 1, wherein total nucleic acid template are correlated with a completed amplification of the nucleic acid template.

3. The method of claim 1, wherein the co-amplifying step is done isothermally.

4. The method of claim 3, wherein the isothermal co-amplifying step comprises nucleic acid sequence-based amplification (NASBA), loop-mediated amplification (LAMP), helicase-dependent amplification (HDA), rolling circle amplification (RCA), recombinase polymerase amplification (RPA), or multiple displacement amplification (MDA).

5. The method of claim 1, wherein analyzing one or more nucleic acid templates comprises testing for aneuploidy, copy number variations (CNVs), and genetic sex, or pathogen detection.

6. (canceled)

7. The method of claim 1, wherein the one or more nucleic acid template is a human chromosome.

8. The method of claim 1, wherein the nucleic acid target is a human chromosome different than the human chromosome of claim 7.

9. The method of claim 7, wherein the human chromosome is Chromosome 21.

10. The method of claim 8, wherein the human chromosome is Chromosome 1.

11. The method of claim 1, wherein the polymerase concentration is from about 0.28 to about 0.56 units/μL.

12. The method of claim 1, wherein the Tm is from about 60° C. to about 65° C. for quantitative LAMP analyses (qLAMP), or from about 57° C. to about 72° C. for nested PCR (NEST).

13. (canceled)

14. The method of claim 1, wherein:
the primer concentration is from about 25 nM to about 1600 nM; or
the reaction mixture comprises a magnesium concentration of from about 5 mM to about 8 mM; or
the nucleic acid template DNA concentration is less than 0.1 ng.

15. (canceled)

16. (canceled)

17. The method of claim 1, wherein a 1.5-fold difference in nucleic acid target DNA concentration is detected.

18. The method of claim 1, further comprising a plurality of primers for amplifying a plurality of nucleic acid targets and a plurality of nucleic acid templates, wherein the plurality of primers comprises one or more primer pairs selected from the group consisting of a F3/B3 primer pair, a LF/LB primer pair, a BIP primer pair, and a FIP primer pair.

19. The method of claim 18, wherein:
the concentrations of the F3/B3 primer pair are from about 25 nM to about 200 nM; or
the concentrations of the LF/LB primer pair are from about 50 nM to about 400 nM: or
the concentration of the BIP primer pair is from about 200 nM to about 1600 nM: or
the concentration of the FIP primer pair is from about 200 nM to about 1600 nM: or
the concentration of the FIP primer pair is from about 100 nM to about 800 nM.

20. (canceled)

21. (canceled)

22. (canceled)

23. The method of claim 12, wherein the FIP primer pair concentration is from about 200 nM to about 1600 nM in SyBr Green based reactions.

24. (canceled)

25. The method of claim 12, wherein the FIP primer pair concentration is from about 100 nM to about 800 nM in probe based reactions.

26. The method of claim 25, wherein the probe-based reaction comprises the presence of from about 100 nM to about 800 nM FIP FQ-Fd duplex


SEQUENCE LISTING (60 sequences)

SEQ ID NO: 1 - DNA, 42 bp, Homo sapiens
tctgcgggaagcaaactgcaataagtatcgacctcgtcggaa

SEQ ID NO: 2 - DNA, 40 bp, Homo sapiens
ccgcttcggtactctgcagcttgagtgtgtggctttcgta

SEQ ID NO: 3 - DNA, 19 bp, Homo sapiens
aggccatgcacagagagaa

SEQ ID NO: 4 - DNA, 19 bp, Homo sapiens
cctagctggtgctccattc

SEQ ID NO: 5 - DNA, 17 bp, Homo sapiens
tcggcagcatcttcgcc

SEQ ID NO: 6 - DNA, 21 bp, Homo sapiens
gaagtgcaactggacaacagg

SEQ ID NO: 7 - DNA, 42 bp, Homo sapiens
cggagctcatggatgctcttcccgcaatttcatccaggacga

SEQ ID NO: 8 - DNA, 40 bp, Homo sapiens
ggctcagctctgcgaccttagtagatgtggccctcaacgg

SEQ ID NO: 9 - DNA, 20 bp, Homo sapiens
gctggggataacgatgagag

SEQ ID NO: 10 - DNA, 18 bp, Homo sapiens
tgctcccgatccacagtg

SEQ ID NO: 11 - DNA, 22 bp, Homo sapiens
ctcatgaatcaaacgcagcact

SEQ ID NO: 12 - DNA, 20 bp, Homo sapiens
cgtcaaggccatcaaggaag

SEQ ID NO: 13 - DNA, 48 bp, Homo sapiens
ccaaagagtaaagtccttctctctcgagagactgttggcccttgaagg

SEQ ID NO: 14 - DNA, 47 bp, Homo sapiens
gtgttgatgttatccaccttttgtggactaggaaaacagatcaatag

SEQ ID NO: 15 - DNA, 20 bp, Homo sapiens
taatcctggaactccggtgc

SEQ ID NO: 16 - DNA, 23 bp, Homo sapiens
tttatgccaattaacattttgac

SEQ ID NO: 17 - DNA, 19 bp, Homo sapiens
atccacagggaggagctct

SEQ ID NO: 18 - DNA, 19 bp, Homo sapiens
ctccacctataaaatcggc

SEQ ID NO: 19 - DNA, 42 bp, Homo sapiens; identical to SEQ ID NO: 1

SEQ ID NO: 20 - DNA, 22 bp, Homo sapiens
attgcagtttgcttcccgcaga

SEQ ID NO: 21 - DNA, 42 bp, Homo sapiens; identical to SEQ ID NO: 7

SEQ ID NO: 22 - DNA, 21 bp, Homo sapiens
ggaagagcatccatgagctcc

SEQ ID NO: 23 - DNA, 21 bp, Homo sapiens
ggaagagcatccatgagctcc

SEQ ID NO: 24 - DNA, 48 bp, Homo sapiens; identical to SEQ ID NO: 13

SEQ ID NO: 25 - DNA, 22 bp, Homo sapiens
agagagaaggactttactcttt

SEQ ID NO: 26 - DNA, 40 bp, Homo sapiens
atcactccacacacgctcggctctggtcttcatcttccgc

SEQ ID NO: 27 - DNA, 41 bp, Homo sapiens
cgactgcaatactcgccagcctgggacacagggaagaactc

SEQ ID NO: 28 - DNA, 20 bp, Homo sapiens
tcaacaagtactccacagcc

SEQ ID NO: 29 - DNA, 20 bp, Homo sapiens
gcatgtcaccaggataagct

SEQ ID NO: 30 - DNA, 20 bp, Homo sapiens
cgtcaccaggtacaccagca

SEQ ID NO: 31 - DNA, 19 bp, Homo sapiens
tgctccaacgtctgctttg

SEQ ID NO: 32 - DNA, 20 bp, Homo sapiens
atcactccacacacgctcgg

SEQ ID NO: 33 - DNA, 21 bp, Homo sapiens
gctctggtcttcatcttccgc

SEQ ID NO: 34 - DNA, 21 bp, Homo sapiens
gctctggtcttcatcttccgc

SEQ ID NO: 35 - DNA, 20 bp, Homo sapiens
ccgagcgtgtgtggagtgat

SEQ ID NO: 36 - DNA, 20 bp, Homo sapiens
gatgatgtcaccagggaagg

SEQ ID NO: 37 - DNA, 20 bp, Homo sapiens
cctcccaggaaaagtggact

SEQ ID NO: 38 - DNA, 38 bp, Homo sapiens
ttgccatgcctgtcacggtgccgctgggattgctgaac

SEQ ID NO: 39 - DNA, 40 bp, Homo sapiens
ggccgggacagttgactagacccatggaaggggtcaacct

SEQ ID NO: 40 - DNA, 21 bp, Homo sapiens
gtccaatcagagctcactgca

SEQ ID NO: 41 - DNA, 23 bp, Homo sapiens
gaaagatggaggggcaggatcag

SEQ ID NO: 42 - DNA, 38 bp, Homo sapiens; identical to SEQ ID NO: 38

SEQ ID NO: 43 - DNA, 20 bp, Homo sapiens
caccgtgacaggcatggcaa

SEQ ID NO: 44 - DNA, 18 bp, Homo sapiens
ggggttcaaaggtgcctg

SEQ ID NO: 45 - DNA, 20 bp, Homo sapiens
cctgtgtgatgtgcatggag

SEQ ID NO: 46 - DNA, 44 bp, Homo sapiens
aggcaagaaaacaaagtctccgagttgagtgccagttctgcttc

SEQ ID NO: 47 - DNA, 43 bp, Homo sapiens
agactcagctttgaacgctgtatgcagctcacccatgcctcag

SEQ ID NO: 48 - DNA, 24 bp, Homo sapiens
ttagtggcttggttctctccttga

SEQ ID NO: 49 - DNA, 25 bp, Homo sapiens
aatcaagcaattaacctcatgggga

SEQ ID NO: 50 - DNA, 44 bp, Homo sapiens; identical to SEQ ID NO: 46

SEQ ID NO: 51 - DNA, 25 bp, Homo sapiens
actcggagactttgttttcttgcct

SEQ ID NO: 52 - DNA, 23 bp, Homo sapiens
ctttaggagagtccgtaatgagg

SEQ ID NO: 53 - DNA, 20 bp, Homo sapiens
ggcttaccgactgaggtcat

SEQ ID NO: 54 - DNA, 38 bp, Homo sapiens
aaggtttagggcctgcgcagtcccactgtcacaagcct

SEQ ID NO: 55 - DNA, 45 bp, Homo sapiens
cttttggccctaaggccttgtcagccttctctttagacttggtca

SEQ ID NO: 56 - DNA, 16 bp, Homo sapiens
tgccatgccccgcaag

SEQ ID NO: 57 - DNA, 22 bp, Homo sapiens
cctccataggctggttctttgc

SEQ ID NO: 58 - DNA, 20 bp, Homo sapiens
aaggtttagggcctgcgcag

SEQ ID NO: 59 - DNA, 38 bp, Homo sapiens; identical to SEQ ID NO: 54

SEQ ID NO: 60 - DNA, 20 bp, Homo sapiens
ctgcgcaggccctaaacctt